(19) United States
(12) Patent Application Publication
(10) Pub. No.: US 2003/0093142 A1
(43) Pub. Date: May 15, 2003

(54) Stent concept for minimization of deployment related wall shear and injury

(76) Inventors: Elazer Edelman, Brookline, MA (US); Kumaran Kolandaivelu, Boston, MA (US)

Correspondence Address: Samuels, Gauthier & Stevens LLP, Suite 3300, 225 Franklin Street, Boston, MA, 02110, US

(21) Appl. No.: 10/271,843

(22) Filed: Oct. 16, 2002

Related U.S. Application Data
(60) Provisional application No. 60/329,628, filed on Oct. 16, 2001.

(51) Int. Cl. A61F 2/06

(52) U.S. Cl. 623/1.15

(57) ABSTRACT

A stent device for minimizing deployment related injuries. The stent device includes at least one structural block of a desired length and includes a plurality of projection struts. The projection struts each have a defined shape, and form a foldable pattern so that the stent device folds onto itself. An expansion mechanism is stored within the structural block. The expansion mechanism unfolds the foldable patterns of the projection struts using expansion so that minimal damage occurs to a vessel wall.

### PRIORITY INFORMATION

This application claims priority from provisional application Ser. No. 60/329,628 filed Oct. 16, 2001.

### BACKGROUND OF THE INVENTION

The invention relates to the field of stents, and in particular a stent for minimization of deployment-related wall shear and injury.

Heart disease is the leading cause of death in the U.S., claiming more than 2 million lives in 1996 alone. Of these, the majority of cases are due to coronary artery disease, or atherosclerosis. Accordingly, extensive effort is spent in the development and use of therapeutic methodologies. Currently, as medicine moves toward less invasive ideals, the use of interventional techniques, such as angioplasty and stenting, has come to the forefront of these efforts. Angioplasty has achieved success, though the benefits are diminished by complications, such as vascular recoil, vasopasm, thrombosis, and long-term restenosis.

To help with some of these acute problems, stenting has been employed, where a structure is expanded, often by balloon angioplasty, at the site of constriction, remaining in place after the catheterization has been finished. Stenting has achieved marked success in allowing for greater post-procedural patency rates, and accordingly has moved from the realm of a bail out device for failed angioplasty procedures to becoming a first-line modality, accounting for over 50% of coronary interventions.

Since their intervention over fifteen years ago, stents have undergone various advances. Structurally, designs have been optimized to allow for radial stiffness to prevent recoil, while allowing axial flexibility for implantation in tortuous vessels. Surface properties and materials have been modified to allow for greater biocompatibility. Improvements in implantation procedures and drug regimens have also helped to reduce both acute and long-term complications.

Additionally, much research is being performed on potential coatings that could allow the stent to serve as a local drug delivery device to further limit its adverse biological presence. Even so, restenosis currently remains a major problem in 30% of the cases, resulting from a migration and over proliferation of smooth muscle cells. Several factors are believed to play a role in this process, from blood bourne mediators, to endothelial damage during the procedure, to deeper vessel injury caused by expansion stresses.

### SUMMARY OF THE INVENTION

According to one aspect of the invention, there is provided a stent device for minimizing and restricting deployment related injuries. The stent device includes at least one structural block of a desired length. The structural block includes a plurality of projection struts. The projection struts each have a defined shape, and form a foldable pattern so that the stent device folds onto itself. An expandable device that is stored within the structural block. The expandable device unfolds the foldable patterns of the projection struts using expansion so that minimal damage occurs to a vessel wall.

According to another aspect of the invention, there is provided a method of deploying a stent to minimize injuries to a vessel wall. The method includes providing at least one structural block of a desired length having a plurality of foldable projection struts. The method also includes providing an expansion mechanism in the structural block. Furthermore, the method includes expanding the expansion mechanism to unfold the foldable projection struts so that minimal damage occurs on the vessel wall

### BRIEF DESCRIPTION OF THE DRAWINGS

FIG. 1 is a minimal diagram of a segment of a stent or a stent illustrating radial expansion and foreshortening leading to shearing wall motions;

FIGS. 2A and 2B are schematic diagrams of a stent in accordance with the invention;

FIG. 3 is a schematic diagram of an equilibrium expansion of a stent in accordance with the invention;

FIG. 4 is a schematic diagram of a stent in accordance with the invention illustrating the cross-sectional limit as the number of struts increase and the length of the struts decrease;

FIG. 5 is a schematic diagram of a cross-sectional face of a stent in accordance with the invention having axial components;

FIGS. 6A-6B are schematic diagrams of structural blocks of a stent in accordance with the invention;

FIGS. 7A-7B are schematics diagrams of secondary struts in a stent in accordance with the invention;

FIGS. 8A-8C are schematic diagrams of various embodiments of flexible links in a stent in accordance with the invention;

FIGS. 9A-9B are schematic diagrams of primary and secondary folds in a stent in accordance with the invention;

FIGS. 10A-10C are schematic diagrams for balloon expansion of a stent in accordance with the invention;

FIG. 11 is a schematic diagram illustrating an alternative technique for expanding a stent in accordance with the invention;

FIG. 12 is a schematic diagram illustrating the maximum formed length of two face struts occurring at end-expansion as one possible mechanism for reducing cross-sectional face area during expansion; and

FIGS. 13A-13C are schematic diagrams demonstrating arrangements for distributing expansion pressures in a stent in accordance with the invention.

### DETAILED DESCRIPTION OF THE INVENTION

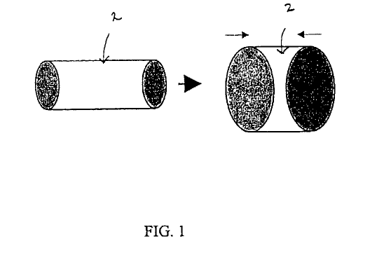

FIG. 1 is a schematic diagram illustrating radial expansion and foreshortening either at the level of a stent, or a section of the stent leading to shearing wall motions. The invention can be used to help minimize and restrict the endothelial damage and deep vessel injury. Currently, it has been shown that a principle cause of vessel injury is due to a shearing action during stent expansion due to the phenomenon of foreshortening. Since struts 2 are made from a stiff material, such as stainless steel, the expansion is taken up by plastic deformations and bending at the strut junctions or the struts 2 themselves, rather than plastic lengthening of the struts 2 themselves. Accordingly, due to the geometric constraints of the inelastic struts 2, as the stent circumference radially increases to that of the desired diameter, the length of a stent section and/or stent must diminish, as shown in FIG. 1. This change in length is a major cause of scraping damage to endothelial cells and deep vessel injury.

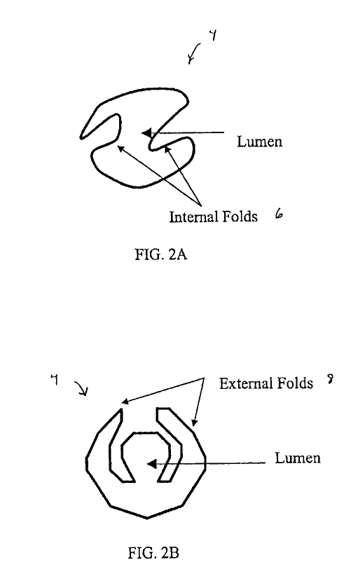

FIGS. 2A and 2B are schematic diagrams of a stent in accordance with the invention. The stent 4 is unique in that upon expansion, it has no axial shortening, thus minimizing the potential for vessel injury. This is to be accomplished generally by acknowledging that the struts are essentially rigid structures, and that symmetric radial expansion will require shortening on the level of a stent segment and/or stent due to geometric constraints, unless stent patterns are specifically made where the stent is folded in on itself, allowing expansion, with the extra surface resulting from these folds rather than length contraction. This folding will also allow the stent to have the reduced cross-sectional profile required for implantation into a stenosed region. Folds 6 or 8 can either be on the interior, as shown in FIG. 2A, or in the exterior, as shown in FIG. 2B of the collapsed stent.

The key feature is that the folded face be projectionable onto a plane perpendicular to the axial direction, and that the expansion of this projection totally describes all significant deformation, either elastic or inelastic, of that face. The cross sectional profile and dynamics of unfolding should be optimized to minimize damage to the vessel wall and maximize utility by considering factors, such as folded cross-sectional profiles, dynamical over-expansion, and dynamical wall stresses.

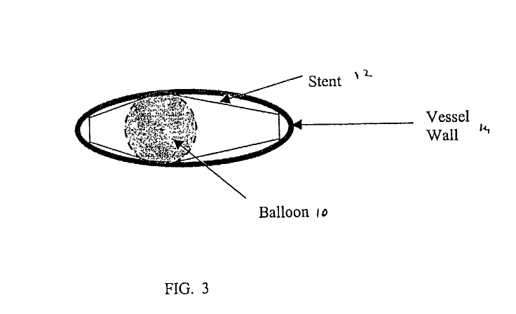

FIG. 3 is a schematic diagram of an equilibrium expansion of the inventive stent 12. Generally, there can be any number of struts of equal or differing lengths, with or without curvature, some portion of which is folded on itself. When fully expanded, the cross-sectional profiles will be dependent on the equilibrium position of the expansion technique, for example, a balloon catheter 10, a stent 12, and a vessel wall 14, and can take on a polygonal-type profile, as shown in FIG. 3.

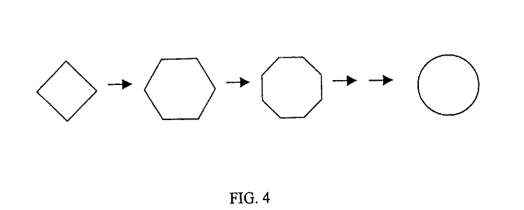

FIG. 4 is schematic diagram illustrating the cross-sectional limit as the number of struts increase and the length of the struts decrease. In the limit of small strut lengths and large strut numbers, this profile becomes circular, as shown in FIG. 4. If curvature is allowed in the struts, curved cross-sectional profiles can also be obtained to allow, among other possibilities, exact circles to be achieved. The cross section characteristics of these struts are dependent on the need for radial support, expandability, and biological compatibility.

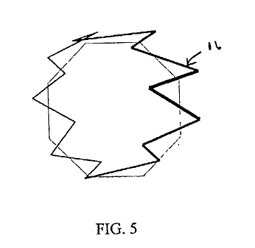

FIG. 5 is a schematic diagram of a cross-sectional face having axial components 16. The cross sectional faces need not be confined to a flat plane, by allowing directional components in the axial direction, as shown in FIG. 5, though such components could result in circumferential shear injury. Thus, they contain a significant axial component 16, as described as that being able to cause significant circumferential shear injury. Also, the significant axial component 16 should not be allowed to contact the vessel wall until end-expansion through hiding on the interior of the unfolding stent.

These cross sectional faces can be projected axially in order to achieve structural blocks of a desired length, being connected by projection struts placed at desired intervals along the cross sectional curve. These junctions can either occur at some, or all of the joints of the faces, or at some other locations on the length of a given face strut. Also, these junctions can be at the same or different locations on the adjoined face, so long as there is no impairment to the required folding pattern. Some patterns, such as pure perpendicular projections with respect to the faces, assure this condition. The principal goals of the projectional struts are to act as spacers between the cross-sectional faces and/or provide longitudinal support for the vessel wall in between the faces, such as the inter-facial regions. As such, there are various considerations that must be made in their placement and design, depending upon their purpose.

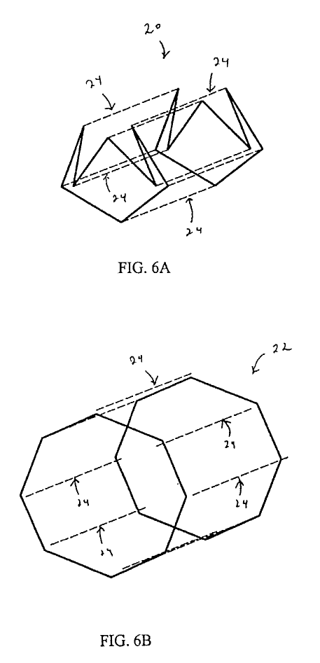

FIGS. 6A-6B are schematic diagrams of structural blocks 20 and 22, respectively. Initially, two principle types of segments can be structural blocks and flexible links. The goal of structural blocks is to provide radial stiffness for some axial distance determined by the length of the block. Accordingly, structural blocks 20 and 22 can be made of two faces held apart by a desired number of projection struts with a minimum of three struts. This minimal number is enforced by the fact that three points determine a plane, thereby imposing a parallel arrangement of the two faces, and a zero curvature condition in-between the faces. Otherwise, the number of struts in structural units is to be determined by a balance between the desire for inter-facial radial support and biological compatibility.

FIG. 6A shows a potential collapsed structural block 20 with eight identical face struts collapsed on itself. FIG. 6B shows an expanded structural block 22 having also eight identical struts. The collapsed structural block 20 includes a number of projection struts 24 indicated by the dashed lines. As previously discussed, these projection struts 24 allow for the overall radial expansions. The expanded structural block 22 also includes projection struts 24 demonstrated by the dashed lines. The projection struts 24 for the expanded structural block 22 provide firm support to the structural block 22 in the axial direction, while providing radial support to the vessel segment stented by the structural block 22.

Likewise, cross sectional characteristics of a given projection strut in a structural block are to be determine by mechanical requirements needed for radial support, biological suitability, and manufacturability. The length of a given projection strut are again dependent on the situation, with longer struts imposing a longer, interfacial, no curvature region, while relying on the strut number and characteristics to provide the requisite cantilevered radial support, rather than the faces themselves. In the limit of zero length, the structural block is minimized to a single face.

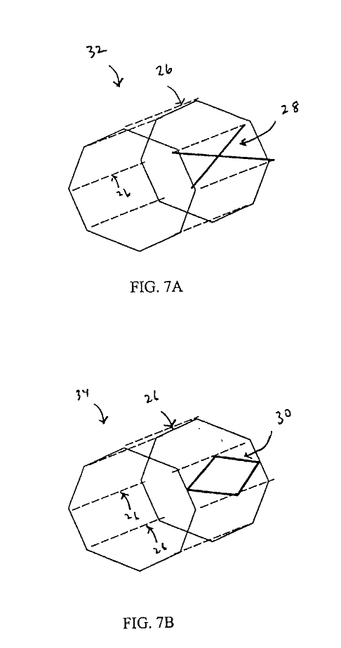

FIGS. 7A-7B are schematics diagrams of secondary struts. Projection struts can be connected to each other by some pattern of secondary struts so as to impose further rigidity on relative facial motions, as shown in FIGS. 7A and 7B. However, it is important that these secondary struts not limit the foldability requirement of the blocks.

FIG. 7A shows a structural block 32 that includes a number of projection struts 26, in this case 8, and a secondary strut 28. Moreover, the structural block 32 is expanded to illustrate the secondary strut 28. Furthermore, the secondary strut 28 is shaped in a criss cross pattern, and is constructed with materials and geometries that will allow the structural block 32 to fold in its collapsed state. The criss cross pattern 28 provides further rigidity on relative facial motions to the structural block 32 and further interfacial radial vessel support.

FIG. 7B shows another structural block 34 that also includes a number of projection struts 26, in this case 8, and a secondary strut 30. Moreover, the structural block 34 is expanded to illustrate the secondary strut 30. Furthermore, the secondary strut 30 is shaped in a diamond pattern, and is constructed with materials that will allow the structural block 34 to fold in its collapsed state. The diamond pattern 30 also provides further rigidity on relative facial motions to the structural block 34 and further interfacial, radial vessel support.

Other patterned shapes can be used to provide rigidity to a structural block, and the structural block can have more than one secondary strut. As discussed previously, the patterned shape(s) should not interfere with the structural block being able to fold.

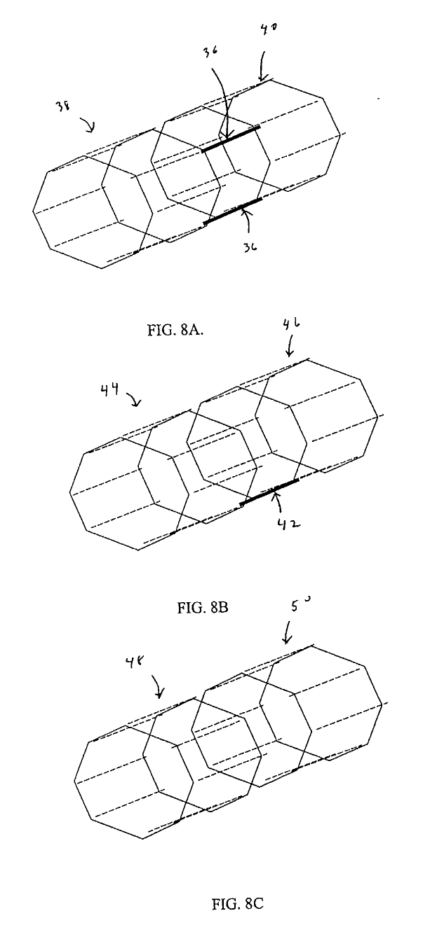

FIGS. 8A-8C are schematic diagrams of various embodiments of flexible links. The flexible links are generally the spaces in-between the structural blocks. Two, one, or zero linking struts are to be used to act as spacers between the structural blocks, placed at some position on the face, joint, or otherwise.

FIG. 8A shows the case of the two linking struts arrangement 36, a line of rotation that is determined by the line connecting the strut/face junctions, which is allowed for a given structural block, as shown in FIG. 8A. In this embodiment, the two flexible linking struts 36 join two eight strut structural blocks 38 and 40. Other structural blocks with different strut arrangements can be used.

FIG. 8B shows the case of the one linking strut arrangement 42, a line of rotation that is determined by the line connecting the strut/face junctions, which is allowed for a given structural block, as shown in FIG. 8B. In this embodiment, the one flexible linking strut 42 joins two eight strut structural blocks 44 and 46, and allows more flexibility and block rotation about a singular point on the strut/face junction. Other structural blocks with different strut arrangements can be used.

FIG. 8C shows the case of the zero linking strut arrangement. In this embodiment, the zero flexible linking struts joins two eight strut structural blocks 48 and 50, and allows for the most flexibility with both rotational and positional freedom, while losing the spacer ability offered by the one and two strut cases. Other structural blocks with different struts arrangement can be also used.

The small number of linking struts means these regions offer little radical support, other than tenting offered by the two bounding structural regions. Accordingly, the lengths of these regions are determined by the need for therapeutic efficacy, such as inter block radial support and biological compatibility. The cross sectional characteristics of these linking struts can be the same or vary from that of the projection struts, the critical design parameters being geared towards achieving the desired flexibility rather than rigidity. Again, in the case of two linking struts, secondary struts can be used to provide extra support against undesirable motions.

Overall, the parameters of these regions used in a given stent can be optimized for various situations to achieve the requirements of radial support while maintaining suitable flexibility.

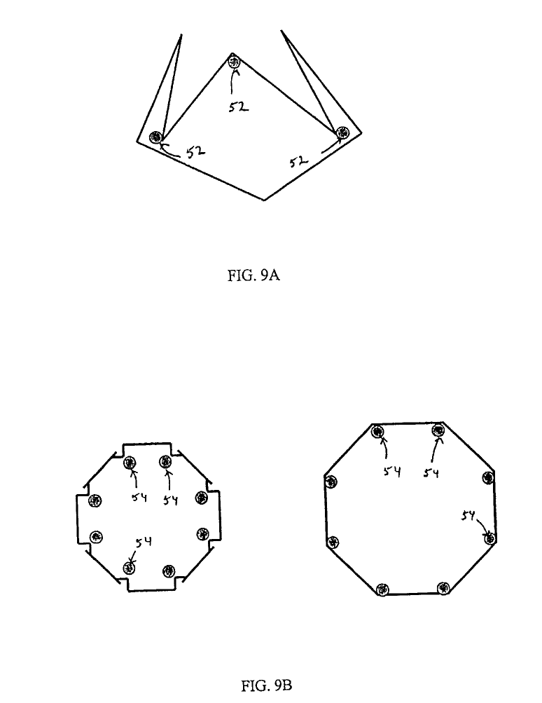

FIGS. 9A-9B are schematic diagrams of primary and secondary folds. One caveat of the longitudinal struts is that upon expansion, as the face opens into its final unfolded state, these struts can contribute circumferential scrapping damage of the vessel wall if they undergo motions that are not purely radial. Though this is the case in current stent modalities as well, the use of specific folds allow for such damage to be eliminated. Two types of folds can be defined as primary folds, as shown in FIG. 9A, and secondary folds, as shown in FIG. 9B.

The purpose of the primary folds 52 is to accommodate the excess area needed in expansion, as mentioned previously. Secondary folds 54 are internal folds, which serve to keep the longitudinal struts, such as projection and/or linking struts, from coming into contact with the vessel wall until a desired point in the expansion process. If this point is chosen as the end of expansion, all procedural shear damage to the vessel wall can be eliminated. One manner in which such action can be achieved is by making the secondary folds 54 sufficiently smaller than the primary folds. Due to the force/lever interactions, the larger primary folds 52 will open first; followed by the secondary folds 54. If the longitudinal struts are placed on these secondary folds 54, they will not come into contact with the vessel wall until the end of the expansion process, as shown in FIG. 9B.

Another possible way in which circumferential wall shear can be avoided is by placing the projectional struts on some suitable location on the primary folds 52 to keep the struts from contacting the wall until the end of the expansion, as shown in FIG. 9A.

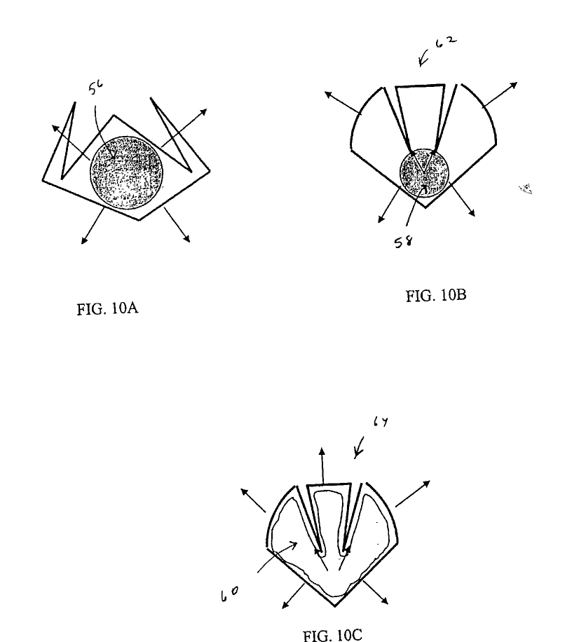

FIGS. 10A-10C are schematic diagrams for balloon expansion. In order to expand the inventive stent, current balloon expansion techniques can be used. However, it is important to consider the expansion dynamics when considering the balloon characteristics and stent design. Certain stents will be unable to expand if their struts are rotated into each other, creating a lock-up condition. Therefore, the balloon 56 and stent 57 must kinematically match for proper expansion, as shown in FIG 10A. If the force offered by the balloon 58 is purely radial along its perimeter, such force must be able to properly unfold the stent 62, as shown in FIG. 10B. If the balloon 60 is more bag-like and can conform to the folded cross-sectional profile of the stent 64, the outward pressure distribution must be able to open the stent 64, as show in FIG. loc.

One can modify a balloon as to maximize the potential benefits of the inventive stent. Since the stents are composed of sequential structural blocks separated by flexible links, these structural blocks can be opened in any combination, particularly if the flexible links have either zero or one linking struts. Therefore, rather than the balloon opening at once along its entire axial length, a design allowing for discrete block expansion will be desirable. This option, in the case of structural blocks connected by no linking struts, can allow for tailoring of the stenting procedures.

After a given segment has be stented, the decision can be made as to whether more structural units are required. For example, the proximal part of a stenosis can be opened, gradually adding structural units until patency is achieved. This is not unlike the current methodology of multiple stenting, only that it can allow for much finer discretizations, and can be performed in the same catheter feed. In order to achieve this, a balloon capable of such discrete opening is required. Also, the balloon should have segments that are able to open to a radial constant diameter for the length of the desired interval. Each balloon segment can also be separated by gaps of minimal length, whose maximum length is that determined by the flexible link regions. One possible way such a balloon can be controlled is to utilize an adjustable sleeve that covers the segments of the balloon that are not to be expanded. Most simply, a sleeve can cover the entire balloon. Once at the site of stenosis, the sleeve can be retracted to the desired amount, allowing the desired number of structural blocks to be opened. Alternatively, the balloon can be divided into discrete chambers, which can be individually pressurized via some sort of valving system.

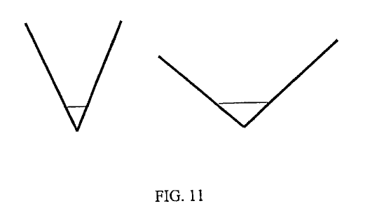

FIG. 11 is a schematic diagram illustrating an alternative technique for expansion. By allowing elastic deformations, which are held in place by some mechanism, such as a sleeve, rather than plastic deformation, a balloon would not be required for full/partial expansion of the stent, since the elastic component would spring open when released from confinement. Furthermore, the struts can be made from shape-retaining materials, such as nitinol, allowing stent expansion through changes in shape, length, and thickness of the stent materials. Upon implantation, the struts can be used to help open the stent. This may be particularly useful for small changes in dimensions close to joints that can yield large changes in the overall shape, as show in FIG. 11, which shows an example of length wise dimension change in strut causing expansion of a joint.

An alternative technique for expanding the stent is to use faces that are made of a shape-retaining material, whose characteristic shape is that of an expanded face. After folding, such a stent can be maneuvered to the proper location, and then triggered by some mechanism to regain its shape either completely, or with the help of some other expansion technique such as a balloon or elastic deformations.

The goal of the stent design is to minimize injury caused during procedural expansions. Such injury has been shown to be a major contributor to post-procedural stent complications, such as thrombosis and restenosis. Modifications in stent design and implantation techniques have already helped limit much of this injury. However, the shearing wall motion imposed upon stent expansion continues to play a significant role in vessel damage.

The described methodology eliminates such undesirable motions, both in the axial and/or circumferential directions, making the only wall interaction a purely radial motion, required for radial support by using cross-sectional folding patterns to account for the extra surface area required for expansion. These folds can be formed by various manufacturing processes, such as extruding pre-folded structural blocks, folding from initially expanded profiles, fold from sheet material or three-dimensional printing of the stent geometry. The use of structural blocks and flexible links allows the overall stent characteristics to be optimized for a given situation, depending of the need for support and flexibility. Furthermore, the use of such segments allows for finer procedural tailoring by discrete block expansions. An important concern in the design of the actual structural blocks is the expansion dynamics. By modifying the mechanism of expansion and stent design, the imposed wall stresses can be reduced to minimize expansion injury. For instance, curved or extra struts can be used to reduce angular wall contact.

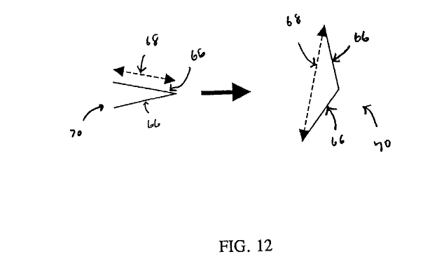

Alternatively, conditions exist, such as where the maximum joint angle, as shown in FIG. 12, between two adjoined face struts 66 occurs at end expansion. This would assure that the largest side 68 of a formed triangle 70 of these two face struts 66 also occurs at end expansion and could be employed to minimize the largest cross-sectional face diameter during expansion.

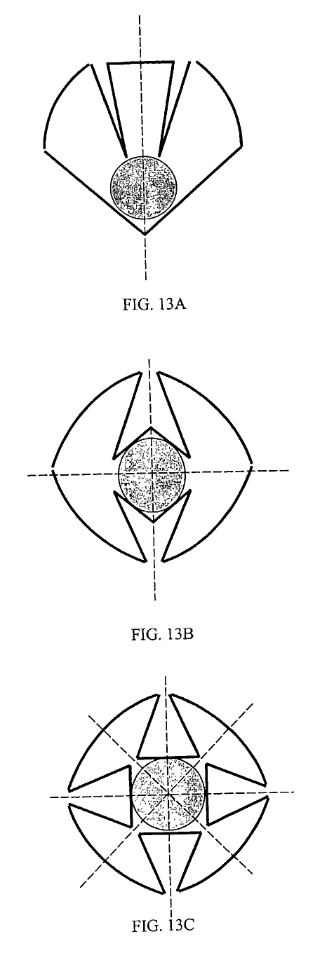

FIGS. 13A-13C are schematic diagrams demonstrating arrangements for distributing expansion pressures. Variations in the symmetric planes of expansion can be made to more evenly distribute the radial expansion pressures, as shown in FIGS. 13A-13C. FIG. 13A shows expansions in one plane of symmetry. FIG. 13B shows expansions in two planes of symmetry. FIG. 13C shows expansions in four planes of symmetry. Other designs can be used for distributing wall stresses associated with expansion in any number of symmetric planes.

The various folded cross-sections, such as elliptical or circular cross-sections, can be tailored to a given individual's stenosed lumin for more suitable implantation, though such utility will also require improved imaging techniques to know the luminal profile in the first place. The concurrent use of the invention and other improvements in stent technology, such as material selection, surface treatments, and drug regimens, could help to further optimize post-procedural outcomes. Additionally, this idea, though described here for use in coronary stents, can also be used for other clinical purposes, such as esophageal, biliary, and general arterial stenting, where expansional injury is a major concern.

Although the present invention has been shown and described with respect to several preferred embodiments thereof, various changes, omissions and additions to the form and detail thereof, may be made therein, without departing from the spirit and scope of the invention.

What is claimed is:

1. A stent device for minimizing and restricting deployment related injuries comprising:
at least one structural block of a desired length including a plurality of projection struts, said plurality of projection struts each having a defined shape and forming a foldable pattern so that said stent device folds onto itself; and
an expansion mechanism that is stored within said structural block, said expansion mechanism unfolding said foldable patterns of said plurality of projection struts using expansion so that minimal damage occurs to a vessel wall.

2. The stent device of claim 1, wherein said at least one structural block is at least three projection struts.

3. The stent device of claim 1 further comprising at least one secondary strut to impose rigidity on relative motions of said projection struts.

4. The stent device of claim 3, wherein said at least one secondary strut is shaped in a criss-cross pattern.

5. The stent device of claim 3, wherein said at least one secondary strut is shaped in a diamond pattern.

6. The stent device of claim 1, wherein said at least one structural block is more than two structural blocks.

7. The stent device of claim 6 further comprising at least one flexible link for coupling said two or more structural blocks.

8. The stent device of claim 1, wherein said expansion mechanism is kinematically matched for proper expansion.

9. A method of deploying a stent to minimize injuries to a vessel wall comprising:
providing at least one structural block of a desired length having a plurality of foldable projection struts;
providing an expandable device in said structural block; and
expanding said expansion mechanism to unfold said foldable projection struts so that minimal damage occurs on said vessel wall;

10. The method of claim 9, wherein said at least one structural block is at least three projection struts.

11. The method of claim 9 further comprising providing at least one secondary strut to impose rigidity on relative motions of said foldable projection struts.

12. The method of claim 11, wherein said at least one secondary strut is shaped in a crisscross pattern.

13. The method of claim 11, wherein said at least one secondary strut is shaped in a diamond pattern.

14. The method of claim 9, wherein said at least one structural block is more than two structural blocks.

15. The method of claim 14 further comprising at least one flexible link for coupling said more than two structural blocks.

16. The method of claim 9, wherein said balloon device is kinematically matched for proper expansion.
